(19) United States
(12) Patent Application Publication
(10) Pub. No.: US 2007/0005015 A1
(43) Pub. Date: Jan. 4, 2007

(54) Disposable syringe

(76) Inventors: Ming-Jeng Shue, Taichung City (TW); Deborah Huang, Taichung City (TW); Phillip Shue, Taichung City (TW)

Correspondence Address: Paul D. Greeley, Esq.;Ohlandt, Greeley, Ruggiero & Perle, L.L.P., 10th Floor, One Landmark Square, Stamford, CT, 06901-2682, US

(21) Appl. No.: 11/320,927

(22) Filed: Dec. 29, 2005

(30) Foreign Application Priority Data
Jun. 13, 2005 (TW) 094119486

(51) Int. Cl. A61M 5/00

(52) U.S. Cl. 604/110; 604/187

(57) ABSTRACT

A disposable syringe includes a needle assembly having a needle seat retained in a barrel by a grip member to fix a needle cannula, a tubular plunger movable in the barrel toward the needle seat during an injection course, and a coupling member having a retained shank in frictional engagement with the plunger, and an anchored portion disposed outwardly of the plunger to engage a rear anchoring portion of the needle seat when the coupling member is moved forwardly. When the plunger is moved further forward to cause the plunger to move relative to the coupling member, the retained shank is released to enable the needle assembly to retract into the plunger by means of a biasing member. A guiding sleeve is sleeved on the needle seat, and can be forced to extend between the needle seat and the grip member so as to decrease the frictional force therebetween for ensuring successful retraction of the needle assembly.

### CROSS-REFERENCE TO RELATED APPLICATION

This application claims priority of Taiwanese publication No. 094119486, filed on Jun. 13, 2005.

### BACKGROUND OF THE INVENTION

1. Field of the Invention

This invention relates to a disposable syringe, more particularly to a disposable syringe with a guiding sleeve for facilitating retraction of a needle into a plunger.

2. Description of the Related Art

Conventional syringes, especially those with a sharp needle, have to be disposed of safely after injection. Therefore, many syringes are provided with a retractable needle that can be retracted into a plunger when the plunger reaches the end of its stroke. In a co-pending U.S. patent application Ser. No. 10/918,020, entitled “Disposable Syringe with a Retractable Needle,” filed by the applicants, there is disclosed a disposable syringe including a barrel which has a retaining region to retain a needle seat for carrying a needle cannula to form a needle assembly, and a friction diminishing region that extends forwardly from the retaining region and that terminates at a shoulder abutment. A plunger is movable in the barrel toward the needle seat for injection. A coupling member frictionally engages the plunger, and has an anchored portion to engage the needle seat. When the needle seat is moved past the friction diminishing region to abut against the shoulder abutment, a subsequent pushing force applied to the plunger results in release of the coupling member from the plunger, thereby enabling the coupling member and the needle assembly to be retracted into the plunger by means of a biasing member. By virtue of the friction diminishing region, the retraction of the needle assembly can be stabilized.

It is desirable to improve the retraction of the syringe when the syringe is adapted for injecting medication of a small volume, i.e., the syringe has a very small inner diameter.

### SUMMARY OF THE INVENTION

The object of the present invention is to provide a disposable syringe which permits steady retraction of a plunger and which ensures successful retraction of a needle assembly even if the syringe is made to have a very small inner diameter.

According to this invention, the disposable syringe includes a needle assembly having a needle seat which includes a front hub portion to fix a needle cannula, a gripped portion, and a rear anchoring portion. A barrel has an inner surrounding barrel surface surrounding an axis in a longitudinal direction to define a passage therein, and including a larger-diameter portion and a smaller-diameter portion. A tubular grip member has an inner surrounding grip surface retainingly sleeved on the gripped portion of the needle seat, and an outer surrounding retained surface that, in a position of use, is engaged with a retaining area of the larger-diameter portion by virtue of a first frictional force generated therebetween.

A tubular plunger is movable in the passage along the larger-diameter portion, and has a front opened end wall which is movable to abut against the tubular grip member, a rear opened end wall which extends outwardly of a rearward opening of the plunger so as to be manually operable, and an intermediate surrounding wall which defines an accommodation chamber. A coupling member has a retained shank which is disposed in the accommodation chamber to be in frictional engagement with the intermediate surrounding wall by virtue of a second frictional force, and an anchored portion which extends from the retained shank forwardly and which is disposed outwardly of the front opened end wall in the position of use so as to be engageable with the rear anchoring portion of the needle seat when the coupling member is moved forwardly. When the grip member is pushed forward by virtue of forward movement of the plunger against the first frictional force, the anchored portion of the coupling member is brought to rub against the rear anchoring portion which remains unmoved in place, so as to be engaged with the rear anchoring portion by a holding force. Once the plunger is moved further forward so as to cause movement of the plunger relative to the coupling member against the second frictional force, the retained shank is released from the intermediate surrounding wall to thereby permit the anchored portion to be moved with the rear anchoring portion to a retracted position where the anchored portion is disposed closer to a rear opened end wall of the plunger and where the needle assembly can be received in the accommodation chamber. A biasing member, such as a coiled spring, is disposed to bias the anchored portion of the coupling member towards the retracted position.

A guiding sleeve is sleeved on the needle seat to permit movement of the needle seat relative thereto, and includes a front resisted end which is disposed to be prevented from moving forward, and an intermediate separating portion which extends rearwardly from the front resisted end to terminate at a rear splitting end. The rear splitting end is interposed between the inner surrounding grip surface of the grip member and the gripped portion of the needle seat. Thus, when the plunger is moved forwards, the intermediate separating portion is forced to extend between the gripped portion and the inner surrounding grip surface so as to decrease the frictional force therebetween.

### BRIEF DESCRIPTION OF THE DRAWINGS

Other features and advantages of the present invention will become apparent in the following detailed description of the preferred embodiments of the invention, with reference to the accompanying drawings, in which:

FIG. 1 is a sectional view of the first preferred embodiment of a disposable syringe according to this invention in a state of use;

FIG. 2 is an exploded sectional view of a needle assembly and a guiding sleeve of the first preferred embodiment;

FIGS. 3 to 5 are fragmentary sectional views of the first preferred embodiment, showing a coupling member in three different states;

FIG. 6 is a sectional view of the first preferred embodiment in a retracted state;

FIGS. 7 and 8 are sectional views of the second preferred embodiment of a disposable syringe according to this invention in a state of use and in a retracted state, respectively;

FIGS. 9 and 10 are sectional views of the third preferred embodiment of a disposable syringe according to this invention in a state of use and in a retracted state, respectively;

FIG. 11 is a fragmentary sectional view of the fourth preferred embodiment of a disposable syringe in a state of use;

FIG. 12 is a fragmentary sectional view of the fifth preferred embodiment of a disposable syringe in a state of use;

FIG. 13 is a cross-sectional view of a grip member of the fifth preferred embodiment taken along lines 13-13 of FIG. 12; and

FIG. 14 is a fragmentary sectional view of the sixth preferred embodiment of a disposable syringe in a state of use;

FIG. 15 is a fragmentary sectional view of the seventh preferred embodiment of a disposable syringe in a state of use; and

FIG. 16 is a fragmentary sectional view of the seventh preferred embodiment of the disposable syringe when a needle seat abuts against a shoulder abutment of a barrel.

### DETAILED DESCRIPTION OF THE PREFERRED EMBODIMENTS

Before the present invention is described in greater detail, it should be noted that same reference numerals have been used to denote like elements throughout the specification.

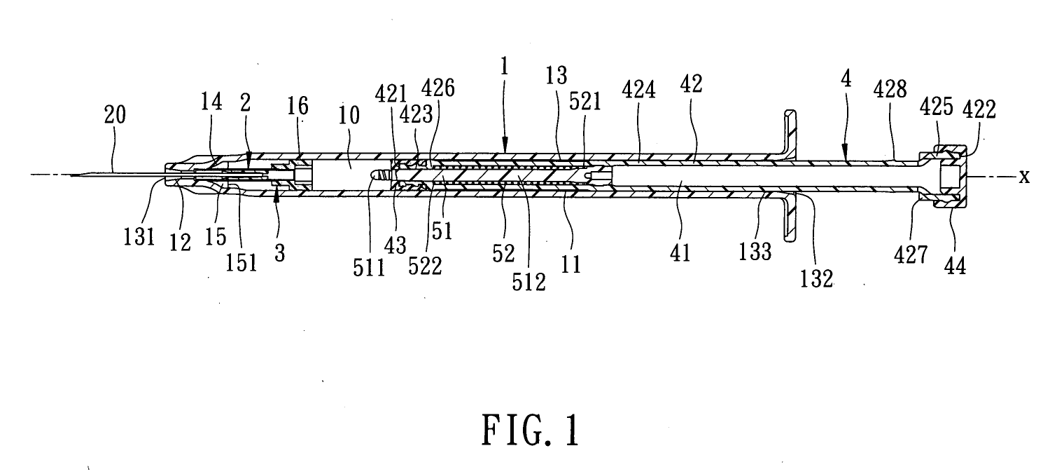

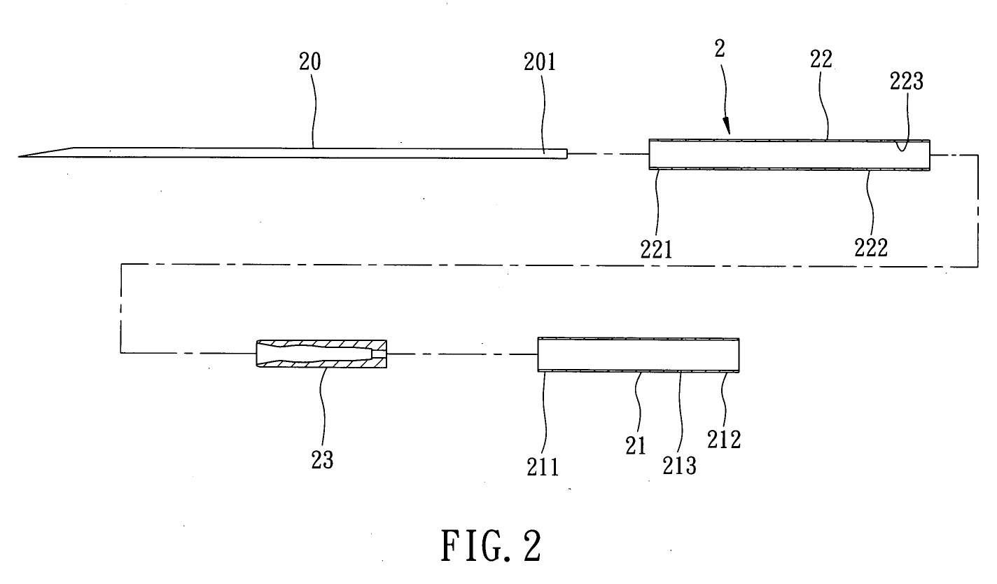

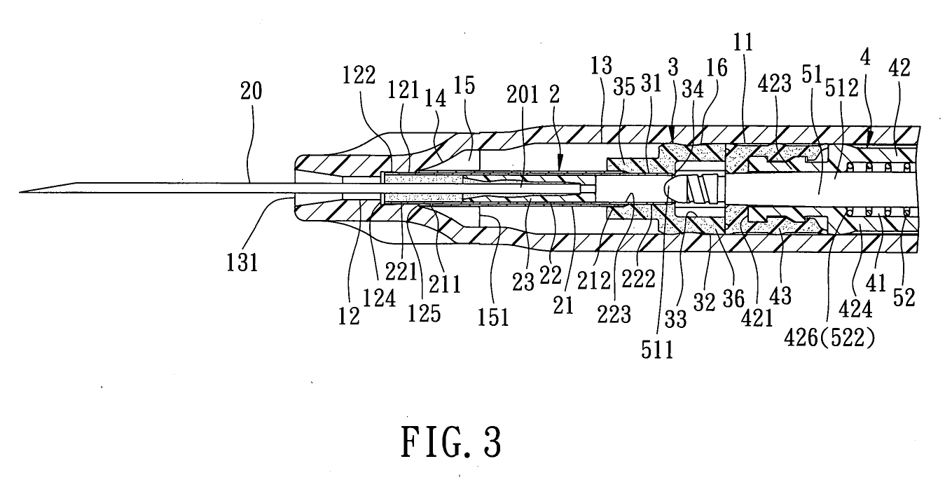

Referring to FIGS. 1 to 3, the first preferred embodiment of a disposable syringe according to the present invention is shown to comprise a needle assembly 2, a barrel 1, a tubular grip member 3, a tubular plunger 4, a coupling member 51, a guiding sleeve 21, and a biasing member 52.

The needle assembly 2 includes a needle cannula 20, a tubular needle seat 22, and a cannula retaining tube 23. The needle seat 22 is in the form of a hard, thin-wall tube, such as a tube of rigid metal, carbon fiber or engineering plastic tube, which includes a front hub portion 221 that is disposed to fix a rear flat-ended segment 201 of the needle cannula 20 therein, a gripped portion 222 which extends from the front hub portion 221 in a longitudinal direction, and a rear anchoring portion 223 which is disposed opposite to the front hub portion 221 in the longitudinal direction. The cannula retaining tube 23 is fitted into the front hub portion 221, and has a tube chamber to receive the needle cannula 20 therein and to secure the flat-ended segment 201 of the needle cannula 20 by means of an adhesive material so as to fix the needle cannula 20 to the needle seat 22.

The barrel 1 has an inner surrounding barrel surface 13 which surrounds an axis (X) in the longitudinal direction, and which defines a passage 10 therein. The passage 10 has rearward and forward openings 132,131 which are disposed opposite to each other in the longitudinal direction. The inner surrounding barrel surface 13 includes a larger-diameter portion 11 and a smaller-diameter portion 12 disposed proximate to the rearward and forward openings 132,131, respectively, and a tapered portion 14 which converges from the larger-diameter portion 11 to the smaller-diameter portion 12.

The larger-diameter portion 11 has a retaining area 16 which is in the form of an annular protrusion and which is spaced apart from the smaller-diameter portion 12 in the longitudinal direction. The smaller-diameter portion 12 has a first shoulder abutment 124, a retaining region 121 which is spaced apart from the first shoulder abutment 124 and which is configured to retain the front hub portion 221 of the needle seat 22 thereat when the needle seat 22 is in a position of use, a friction diminishing region 122 which is disposed between the first shoulder abutment 124 and the retaining region 121, and a second shoulder abutment 125 which is disposed between the retaining region 121 and the retaining area 16. The passage 10 at the friction diminishing region 122 has a diameter larger than that of the passage 10 at the retaining region 121. The tapered portion 14 has a plurality of ribs 15 which are angularly displaced from one another about the axis (X) and which have rearwardly facing ends 151.

The grip member 3 includes a small front segment 35 which has an inner surrounding grip surface 31 that is retainingly sleeved on the gripped portion 222 of the tubular needle seat 22, and a large rear segment 36 which is opposite to the small front segment 35 along the axis (X) and which has an outer surrounding retained surface 32 that, in the position of use, is engaged with the retaining area 16 by virtue of a first frictional force generated therebetween, and an inner surrounding surface 33 opposite to the outer surrounding retained surface 32 radially.

The plunger 4 is disposed to be movable in the passage 10 along the larger-diameter portion 11, and has a front opened end wall 421 which is movable to abut against the grip member 3, a rear opened end wall 422 which is disposed opposite to the front opened end wall 421 and which extends outwardly of the rearward opening 132 so as to be manually operable, and an intermediate surrounding wall 42 which is interposed between the front and rear opened end walls 421,422 and which defines an accommodation chamber 41. The intermediate surrounding wall 42 has a smaller front segment 423 and a larger rear segment 424 so as to form a shoulder 426 therebetween. An enlarged terminal segment 425 is disposed between the larger rear segment 424 and the rear opened end wall 422. An end cap 44 is disposed to cover the rear opened end wall 422. A seal ring 43, which is made of a deformable material, is sleeved retainingly on an outer peripheral surface of the smaller front segment 423 so as to be slidable on and to be in fluid-tight frictional engagement with both the larger-diameter portion 11 of the inner surrounding barrel surface 13 and the plunger 4.

The coupling member 51 has a retained shank 512 which extends along the axis (X) and which is disposed in the accommodation chamber 41 to be in frictional engagement with the intermediate surrounding wall 42 by virtue of a second frictional force, and an anchored portion 511 which extends from the retained shank 512 forwardly and which is disposed outwardly of the seal ring 43 in the position of use so as to be engageable with the rear anchoring portion 223 of the needle seat 22 when the coupling member 51 is moved forwardly towards the forward opening 131.

The guiding sleeve 21 is shorter than the needle seat 22, is made from a rigid material, such as a rigid metal, carbon fiber or engineering plastic material, and is sleeved on the needle seat 22 to permit movement of the needle seat 22 relative thereto. The guiding sleeve 21 includes a front resisted end 211 which is disposed to abut against the second shoulder abutment 125 to be prevented from moving towards the forward opening 131, and an intermediate separating portion 213 which extends rearwardly from the front resisted end 211, and which terminates at a rear splitting end 212. The rear splitting end 212 is interposed between the inner surrounding grip surface 31 and the gripped portion 222. Preferably, a retaining protrusion 34 integrally formed with the small front segment 35 of the grip member 3 is provided to retain the rear splitting end 212.

The biasing member 52 is a coiled spring surrounding the retained shank 512 of the coupling member 51, and has a front spring end 522 which abuts against the shoulder 426, and a rear spring end 521 which is retained on a rear end of the retained shank 512 such that the coiled spring is compressed in the position of use.

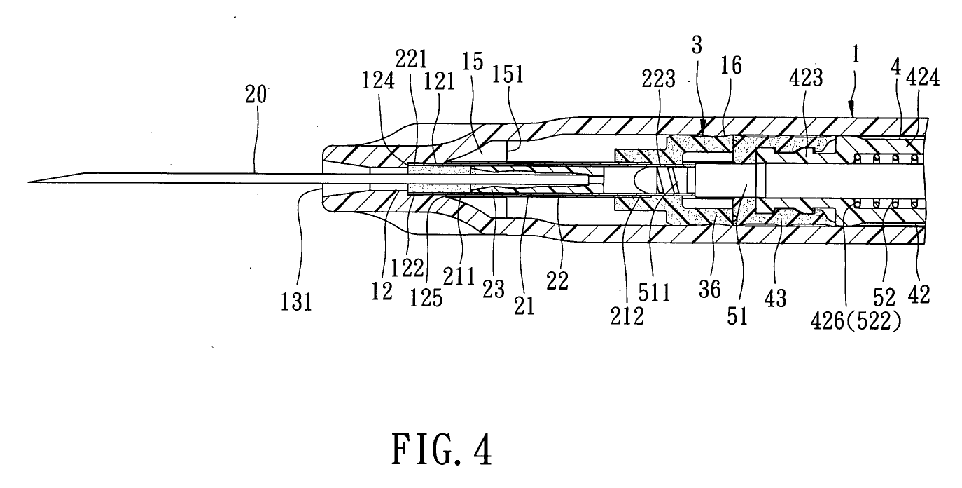

As shown in FIG. 4, after completion of an injection course, the plunger 4 is pressed forwardly by a pushing force to permit abutment of the seal ring 43 against the grip member 3. At this time, the anchored portion 511 is extended into the rear segment 36. Subsequently, a manual pushing force is further applied to the plunger 4 to push the grip member 3 forward against the first frictional force (i.e., the frictional engagement between the grip member 3 and the retaining area 16) such that the anchored portion 511 is partially extended into and is engaged with the rear anchoring portion 223, and such that the front hub portion 221 of the needle seat 22 moves past the friction diminishing region 122 to abut against the first shoulder abutment 124 to thereby provide a triggering action for retraction of the needle cannula 20 (to be described in detail hereinafter). Hence, the movement of the front hub portion 221 results in reduced friction between the front hub portion 221 and the smaller-diameter portion 12.

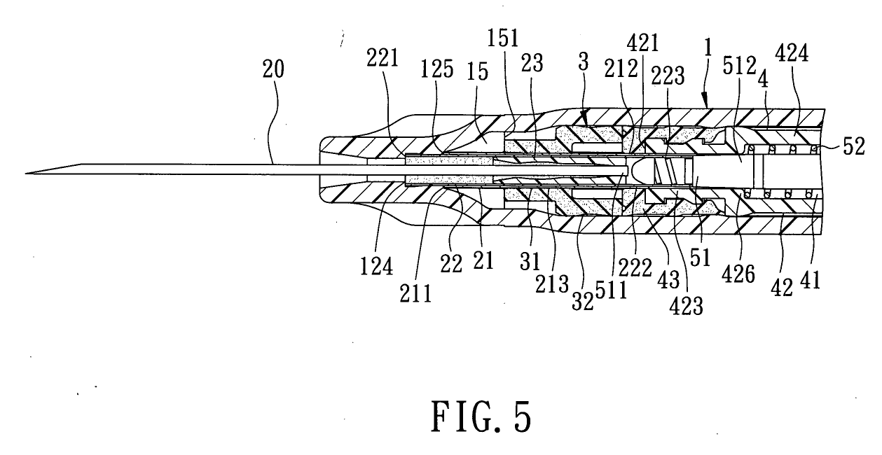

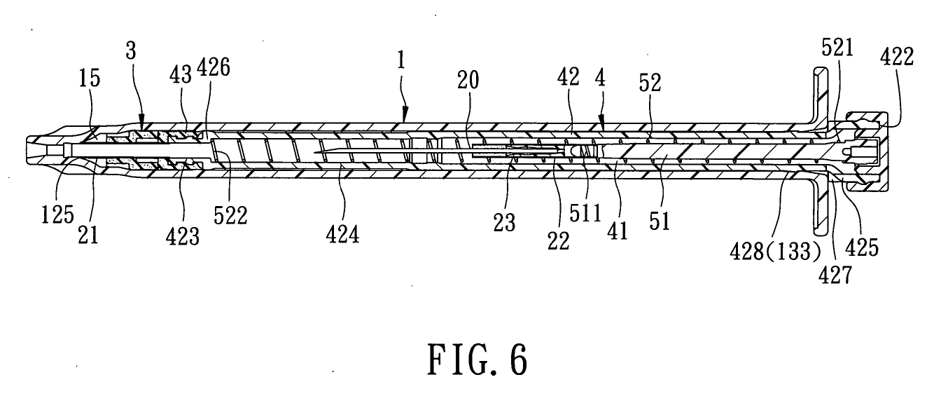

As shown in FIG. 5, when a pushing force is subsequently applied to the plunger 4, the anchored portion 511 rubs against the rear anchoring portion 223, which remains unmoved in place due to abutment of the front hub portion 221 against the first shoulder abutment 124, so as to be engaged with the rear anchoring portion 223 by a holding force. Meanwhile, the grip member 3 is brought to abut against the rearwardly facing ends 151 of the ribs 15. At the same time, the intermediate separating portion 213 extends between the gripped portion 222 of the needle seat 22 and the inner surrounding grip surface 31 of the grip member 3 so as to decrease the frictional force between the gripped portion 222 and the inner surrounding grip surface 31. Besides, the rear splitting end 212 of the guiding sleeve 21 extends in the seal ring 43 and the front opened end wall 421 of the plunger 4 to thereby force the intermediate surrounding wall 42 to yield radially and outwardly so as to further decrease the second frictional force. Furthermore, the plunger 4 is moved forward relative to the coupling member 51 against the second frictional force such that the retained shank 512 is released from the intermediate surrounding wall 42. Thus, the anchored portion 511, as well as the needle assembly 2 held by the holding force (including the needle seat 22 and the needle cannula 20), is moved by the biasing force of the biasing member 52 to a retracted position, as shown in FIG. 6, where the anchored portion 511 is disposed closer to the rear opened end wall 422 of the plunger 4 so as to enable the needle assembly 2 to be received in the accommodation chamber 41.

As illustrated, during retraction of the needle assembly 2, the guiding sleeve 21 is extended between the gripped portion 222 of the needle seat 22 and the inner surrounding grip surface 31 of the grip member 3 so as to decrease the frictional force therebetween, and further extends in the seal ring 43 and the front opened end wall 421 of the plunger 4 to thereby force the intermediate surrounding wall 42 to yield radially and outwardly so as to further decrease the second frictional force. Besides, the friction diminishing region 122 provides a space of the triggering action for retraction of the needle assembly 2 so that the friction between the needle seat 22 and the smaller-diameter portion 12 can be reduced. Therefore, successful and smooth retraction of the needle assembly 2 can be ensured.

In other words, with such a construction, the needle seat 22 can be configured to have a relatively small diameter, and thus the needle cannula 20 can be formed to have a relatively small diameter so as to be adapted for injecting medication of a very small volume, such as 1 ml. Likewise, the barrel 1 can be configured to have a smaller diameter with a relatively compensatory elongation of the length of the barrel 1 for a unit medication volume. Thus, the spacing of graduations (not shown) marked on the barrel 1 of extremely small volume, such as 0.01 cc., can be relatively large to facilitate accurate reading of the volume of medication in an injection course. Furthermore, the cannula retaining tube 23 can be used as a medium to attach the needle cannula 20 to the needle seat 22 so that the assembly of the needle cannula 20 and the needle seat 22 is convenient to conduct, and the connection between the needle cannula 20 and the needle seat 22 is firm and steady.

It is noted that a rib 427 is disposed on the enlarged terminal segment 425, and is configured to abut against a rear end of the larger-diameter portion 11, as shown in FIG. 6, so as to prevent insertion of the enlarged terminal segment 425 into the barrel 1 to thereby prevent undesired deformation of the barrel 1.

Moreover, as shown in FIGS. 1 and 6, a retaining groove 133 is formed in the larger-diameter portion 11 of the barrel 1 adjacent to the rearward opening 132. A retaining protrusion 428 is disposed on the larger rear segment 424 adjacent to the enlarged terminal segment 425, and is configured to engage the retaining groove 133 to prevent undesired rearward pulling of the plunger 4.

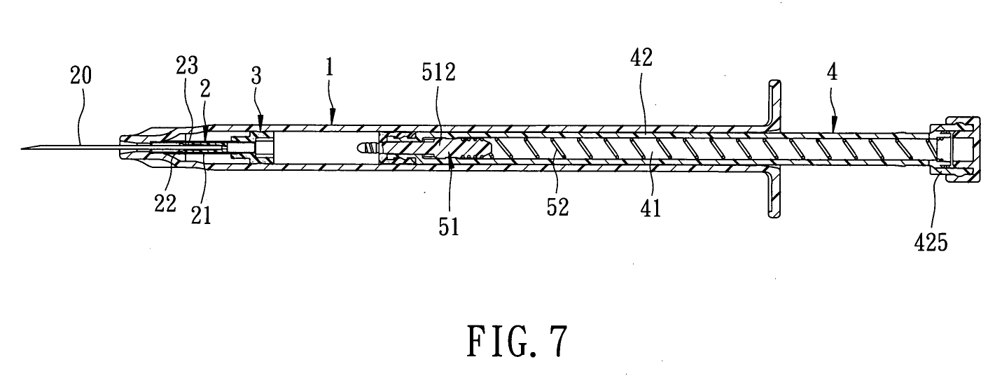

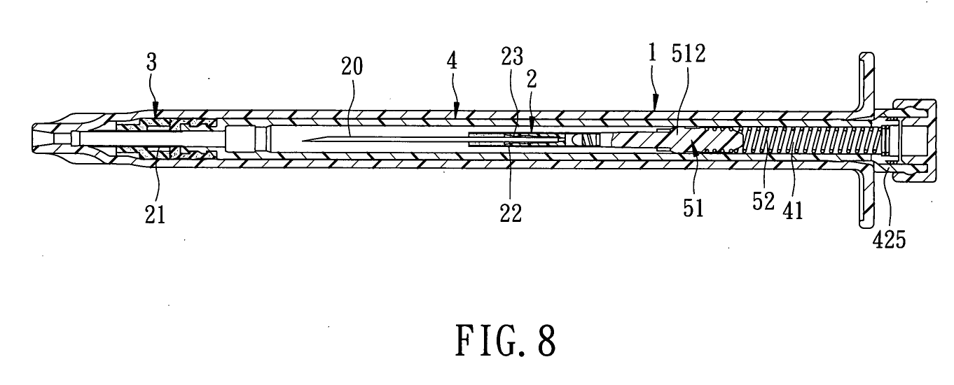

Referring to FIGS. 7 and 8, the second preferred embodiment of a disposable syringe according to this invention is similar to the first preferred embodiment in construction and function, except that the biasing member 52, in the form of a coiled spring, is connected to the coupling member 51 and the enlarged terminal segment 425 of the plunger 4 such that the biasing member 52 is tensioned when the retained shank 512 is in frictional engagement with the intermediate surrounding wall 42 by virtue of the second frictional force.

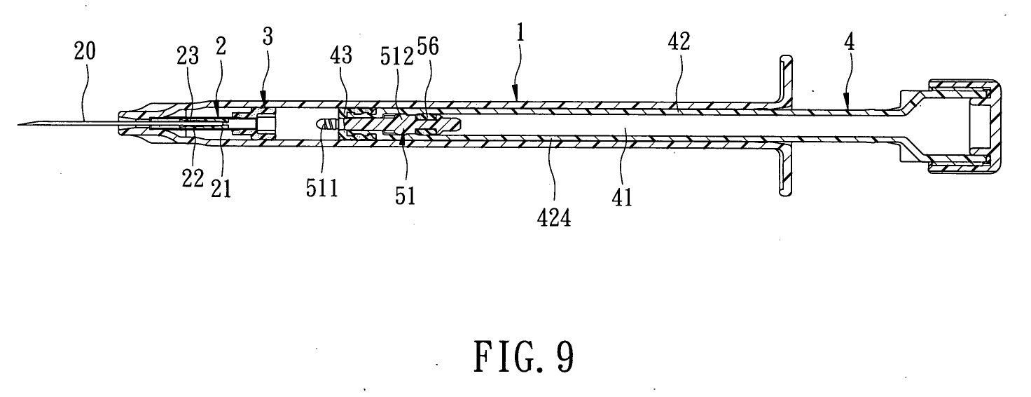

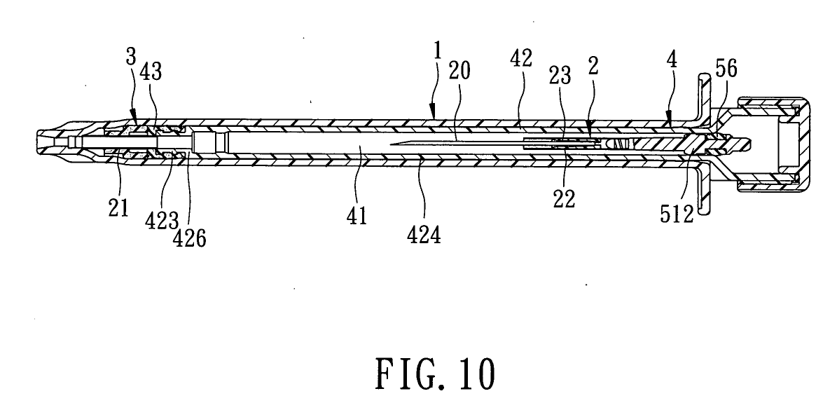

Referring to FIGS. 9 and 10, the third preferred embodiment of a disposable syringe according to this invention is similar to the first preferred embodiment in construction. The difference resides in that the biasing member, instead of being formed as a coiled spring as in the previous embodiments, includes a fluid, such as air, which is contained in the accommodation chamber 41 in the tubular plunger 4 at a relatively reduced pressure, and a sealing member 56 which is sleeved on the retained shank 512 of the coupling member 51 to provide a seal between the coupling member 51 and the intermediate surrounding wall 42 so as to trap the fluid in the accommodation chamber 41. Thus, when the retained shank 512 is released from the intermediate surrounding wall 42, the anchored portion 511 is suctioned to the retracted position due to a pressure difference between the ambient atmosphere and the reduced pressure.

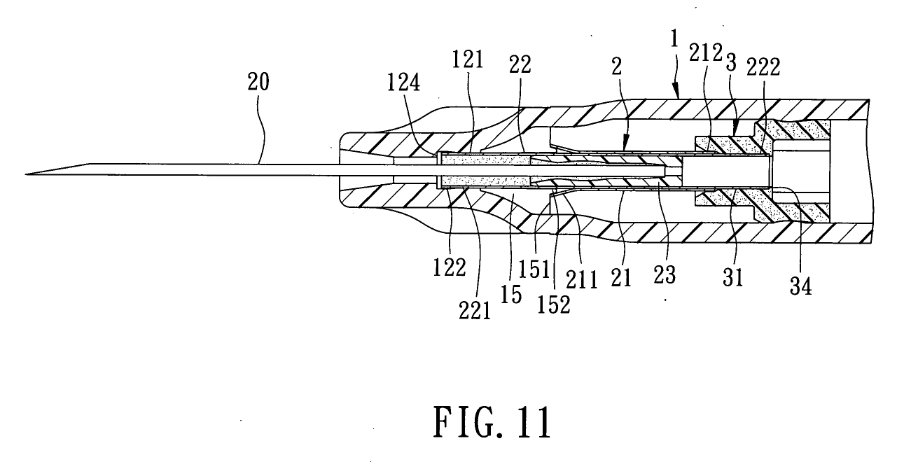

Referring to FIG. 11, the fourth preferred embodiment of a disposable syringe according to this invention is similar to the previous embodiments. In this embodiment, the rearwardly facing ends 151 of the ribs 15 have an annular protrusion 152 surrounding the front hub portion 221 of the needle seat 22. In addition, the front resisted end 211 of the guiding sleeve 21 is flared outwardly and radially and is sleeved on the annular protrusion 152 such that forward movement of the front resisted end 211 is prevented by the rearwardly facing ends 151.

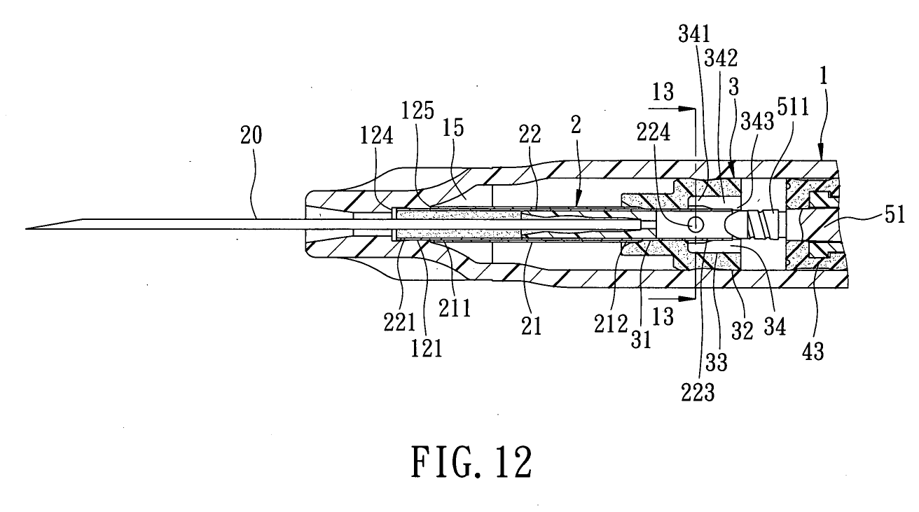

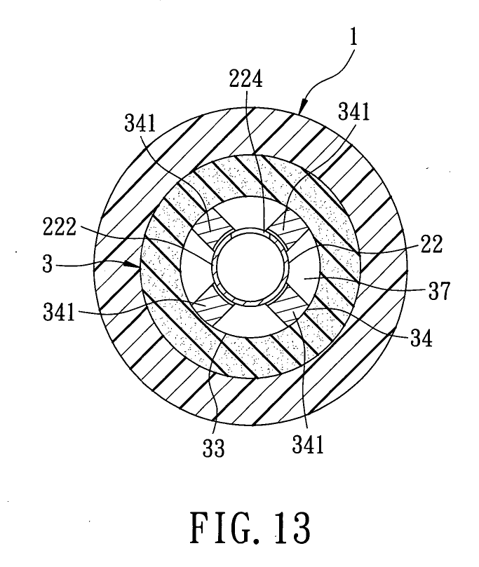

Referring to FIGS. 12 and 13, the fifth preferred embodiment of a disposable syringe according to this invention is similar to the first, second and third preferred embodiments in construction. The differences reside in that the inner surrounding surface 33 of the grip member 3 is spaced apart from the rear anchoring portion 223 of the needle seat 22 radially, and has a plurality of clamp ribs 34 which extend from the inner surrounding surface 33 radially toward the axis (X) and which are angularly displaced from one another to define a plurality of spaces 37 thereamong. Each of the clamp ribs 34 has a front segment 341 slightly spaced apart from the gripped portion 222 of the needle seat 22, a rear segment 342 which retains the grip portion 222, and a block segment 343 which blocks the needle seat 22. Further, the rear anchoring portion 223 of the needle seat 22 has a through hole 224 which extends radially and which is communicated with one of the spaces 37 such that injection liquid remaining between the seal ring 43 and the grip member 3 can flow through the spaces 37 and the through hole 224 into the needle cannula 20, thereby minimizing the amount of residual injection liquid in the plunger 4.

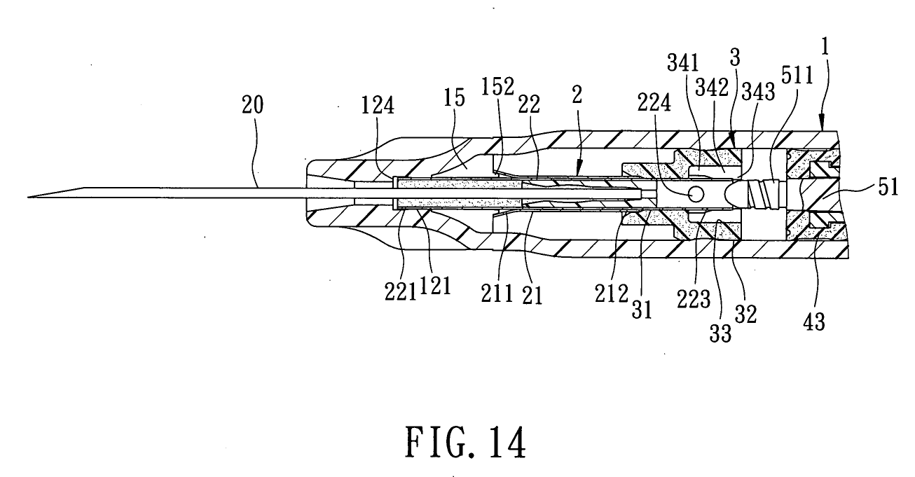

Referring to FIG. 14, the sixth preferred embodiment of a disposable syringe according to this invention has the additional features described in the fourth and fifth preferred embodiments. That is, the ribs 15 have an annular protrusion 152 for retaining the front resisted end 211 of the guiding sleeve 21. The grip member 3 has a plurality of clamp ribs 34 to define spaces thereamong, and the needle seat 22 has a through hole 224 intercommunicating the spaces so as to facilitate flow of injection liquid into the needle seat 22.

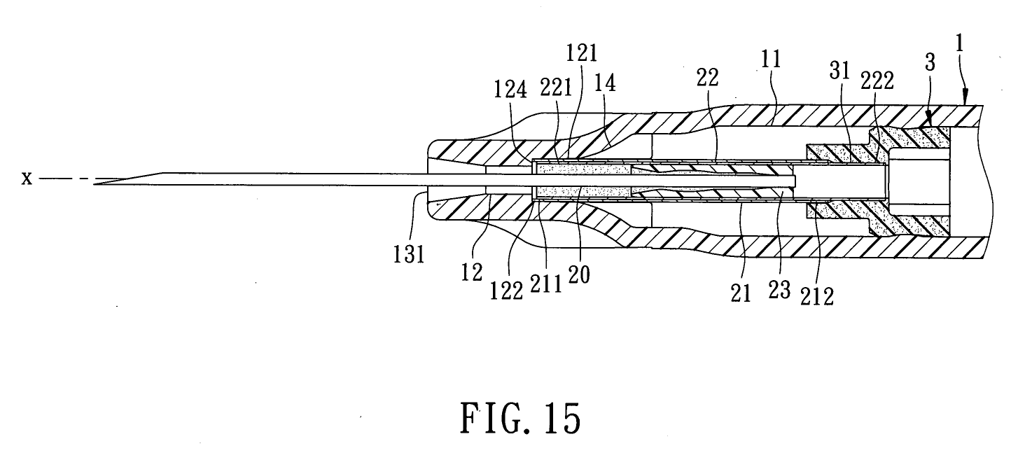

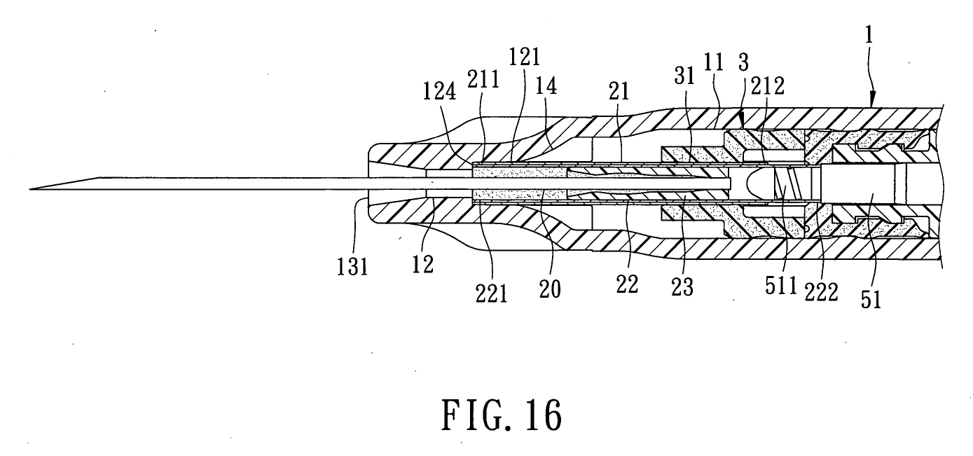

Referring to FIGS. 15 and 16, the seventh preferred embodiment of a disposable syringe according to this invention is similar to the first, second, third and fifth preferred embodiments in construction. The differences reside in that the front resisted end 211 of the guiding sleeve 21 is disposed to abut against the first shoulder abutment 124 so as to prevent from being moved forwardly. Hence, the second shoulder abutment 125 is not required.

While the present invention has been described in connection with what is considered the most practical and preferred embodiments, it is understood that this invention is not limited to the disclosed embodiments but is intended to cover various arrangements included within the spirit and scope of the broadest interpretations and equivalent arrangements.

We claim:

1. A disposable syringe comprising:
a needle cannula;
a tubular needle seat including a front hub portion which is disposed to fix said needle cannula therein, a gripped portion which extends from said front hub portion in a longitudinal direction, and a rear anchoring portion which is disposed opposite to said front hub portion in the longitudinal direction;
a barrel having an inner surrounding barrel surface which surrounds an axis in the longitudinal direction, and which defines a passage therein, said passage having rearward and forward openings which are disposed opposite to each other in the longitudinal direction, said inner surrounding barrel surface including a larger-diameter portion and a smaller-diameter portion which are disposed proximate to said rearward and forward openings, respectively, said larger-diameter portion having a retaining area which is spaced apart from said smaller-diameter portion in the longitudinal direction, said smaller-diameter portion having a first shoulder abutment for abutment of said front hub portion thereagainst;
a tubular grip member which has an inner surrounding grip surface that is retainingly sleeved on said gripped portion of said tubular needle seat, and an outer surrounding retained surface that, in a position of use, is engaged with said retaining area by virtue of a first frictional force generated therebetween;
a tubular plunger which is disposed to be movable in said passage along said larger-diameter portion, said plunger having a front opened end wall which is movable to abut against said tubular grip member, a rear opened end wall which is disposed opposite to said front opened end wall and which extends outwardly of said rearward opening so as to be manually operable, and an intermediate surrounding wall which is interposed between said front and rear opened end walls and which defines an accommodation chamber;
a coupling member having a retained shank which extends along the axis and which is disposed in said accommodation chamber to be in frictional engagement with said intermediate surrounding wall by virtue of a second frictional force, and an anchored portion which extends from said retained shank forwardly and which is disposed outwardly of said front opened end wall in the position of use so as to be engageable with said rear anchoring portion when said coupling member is moved forwardly towards said forward opening such that, when said grip member is pushed forward by virtue of forward movement of said plunger against the first frictional force, said anchored portion is brought to rub against said rear anchoring portion, which remains unmoved in place due to abutment of said front hub portion against said first shoulder abutment, so as to engage said rear anchoring portion by a holding force, and such that, once said plunger is moved further forward so as to cause movement of said plunger relative to said coupling member against the second frictional force, said retained shank is released from said intermediate surrounding wall to thereby permit said anchored portion to be moved with said rear anchoring portion by the holding force to a retracted position where said anchored portion is disposed closer to said rear opened end wall and where said needle seat and said needle cannula are received in said accommodation chamber;
a guiding sleeve sleeved on said needle seat to permit movement of said needle seat relative thereto, said guiding sleeve including a front resisted end which is disposed to be prevented from moving towards said forward opening, and an intermediate separating portion which extends rearwardly from said front resisted end, and which terminates at a rear splitting end, said rear splitting end being interposed between said inner surrounding grip surface and said gripped portion such that, when said plunger is moved forwards, said intermediate separating portion is forced to extend between said gripped portion and said inner surrounding grip surface so as to decrease the frictional force between said gripped portion and said inner surrounding grip surface; and
a biasing member disposed to bias said anchored portion towards the retracted position.

2. The disposable syringe of claim 1, wherein said smaller-diameter portion has a retaining region which is spaced apart from said first shoulder abutment and which is configured to retain said front hub portion thereat when said needle seat is in the position of use, and a friction diminishing region which is disposed between said first shoulder abutment and said retaining region such that, when said grip member is pushed forward by virtue of the forward movement of said plunger against the first frictional force, said front hub portion is moved past said friction diminishing region to abut against said first shoulder abutment to thereby provide a triggering action for retraction of said needle cannula.

3. The disposable syringe of claim 2, wherein said passage at said friction diminishing region has a diameter larger than that of said passage at said retaining region.

4. The disposable syringe of claim 1, wherein said smaller-diameter portion has a second shoulder abutment which is disposed between said first shoulder abutment and said retaining area and which is disposed to prevent said front resisted end of said guiding sleeve from being moved forwardly.

5. The disposable syringe of claim 1, wherein said inner surrounding barrel surface further includes a tapered portion which converges from said larger-diameter portion to said smaller-diameter portion and which has a plurality of ribs that are angularly displaced from one another about the axis and that have rearwardly facing ends such that once said grip member is brought to abut against said rearwardly facing ends, said anchored portion of said coupling member is permitted to engage said rear anchoring portion of said needle seat by the holding force, thereby enabling said intermediate separating portion of said guiding sleeve to extend between said gripped portion and said inner surrounding grip surface.

6. The disposable syringe of claim 5, wherein said rearwardly facing ends have an annular protrusion surrounding said front hub portion, said front resisted end of said guiding sleeve being flared outwardly and radially to be sleeved on said annular protrusion such that forward movement of said front resisted end is prevented by said rearwardly facing ends.

7. The disposable syringe of claim 1, wherein said rear splitting end of said guiding sleeve extends in said front opened end wall of said plunger to thereby force said intermediate surrounding wall to yield radially and outwardly so as to further decrease the second frictional force when said intermediate separating portion is forced to extend between said gripped portion and said inner surrounding grip surface.

8. The disposable syringe of claim 1, wherein each of said needle seat and said guiding sleeve is made from a rigid material.

9. The disposable syringe of claim 1, further comprising a cannula retaining tube which is fitted into said front hub portion and which has a tube chamber to receive said needle cannula and an adhesive material that is disposed to adhere said needle cannula to said cannula retaining tube.

10. The disposable syringe of claim 1, wherein said grip member includes a small front segment which has said inner surrounding grip surface, and a large rear segment which is opposite to said small front segment along the axis and which has said outer surrounding retained surface, an inner surrounding surface that is spaced apart from said rear anchoring portion of said needle seat radially and that is radially opposite to said outer surrounding retained surface, and a plurality of clamp ribs that extend from said inner surrounding surface radially toward the axis and that are angularly displaced from one another to define a plurality of spaces thereamong, said rear anchoring portion of said needle seat having a through hole which extends radially and which is communicated with one of said spaces so as to facilitate the inflow of injection liquid into said needle seat.

11. The disposable syringe of claim 1, wherein said front resisted end of said guiding sleeve is disposed to abut against said first shoulder abutment so as to prevent from being moved forwardly.
